(19) United States
(12) Patent Application Publication
(10) Pub. No.: US 2008/0044896 A1
(43) Pub. Date: Feb. 21, 2008

(54) Novel human secreted proteins and polynucleotides encoding the same

(76) Inventors: Gregory Donoho, Indianapolis, IN (US); Carl Johan Friddle, The Woodlands, TX (US); Glenn Friedrich, Houston, TX (US); Erin Hilbun, Denton, TX (US); Yi Hu, Spring, TX (US); Brian Mathur, Nashville, TN (US); Maricar Miranda, The Woodlands, TX (US); Michael C. Nehls, Stockdorf (DE); Nghi D. Nguyen, Sugar Land, TX (US); Arthur T. Sands, The Woodlands, TX (US); John Scoville, Pearland, TX (US); C. Alexander Turner JR., The Woodlands, TX (US); D. Wade Walke, Spring, TX (US); Xiaoming Wang, Burr Ridge, IL (US); Frank Wattler, Muensing (DE); Nathaniel L. Wilganowski, Spring, TX (US); Qiongshu Xie, Needham, MA (US); Xuanchuan Yu, Conroe, TX (US); Brian Zambrowicz, The Woodlands, TX (US)

Correspondence Address: LEXICON PHARMACEUTICALS, INC., 8800 TECHNOLOGY FOREST PLACE, THE WOODLANDS, TX, 77381-1160, US

(21) Appl. No.: 11/354,210

(22) Filed: Feb. 13, 2006

Related U.S. Application Data
(63) Continuation-in-part of application No. 10/901,801, filed on Jul. 29, 2004, now abandoned.
Application No. 10/901,801 is a continuation of application No. 09/667,380, filed on Sep. 22, 2000, now abandoned.
(63) Continuation-in-part of application No. 09/689,911, filed on Oct. 11, 2000, now abandoned.
(63) Continuation-in-part of application No. 10/999,215, filed on Nov. 29, 2004, now abandoned.
Application No. 10/999,215 is a continuation of application No. 09/691,343, filed on Oct. 18, 2000, now abandoned.
(63) Continuation-in-part of application No. 11/285,738, filed on Nov. 22, 2005, now abandoned.
Application No. 11/285,738 is a continuation of application No. 09/714,883, filed on Nov. 16, 2000, now abandoned.
(63) Continuation-in-part of application No. 09/863,823, filed on May 23, 2001.
(63) Continuation-in-part of application No. 11/039,362, filed on Jan. 19, 2005.
Application No. 11/039,362 is a continuation of application No. 09/898,456, filed on Jul. 3, 2001, now abandoned.
(63) Continuation-in-part of application No. 09/899,514, filed on Jul. 5, 2001, now abandoned.
(63) Continuation-in-part of application No. 10/972,984, filed on Oct. 25, 2004, now abandoned.
Application No. 10/972,984 is a continuation of application No. 09/952,474, filed on Sep. 12, 2001, now abandoned.
(63) Continuation-in-part of application No. 11/049,637, filed on Feb. 2, 2005, now abandoned.
Application No. 11/049,637 is a continuation of application No. 09/953,096, filed on Sep. 14, 2001, now Pat. No. 6,867,291.
(63) Continuation-in-part of application No. 11/012,588, filed on Dec. 15, 2004, now abandoned.
Application No. 11/012,588 is a continuation of application No. 09/957,832, filed on Sep. 21, 2001, now abandoned.
(63) Continuation-in-part of application No. 10/901,803, filed on Jul. 29, 2004, now abandoned.
Application No. 10/901,803 is a continuation of application No. 09/962,740, filed on Sep. 25, 2001, now abandoned.
(63) Continuation-in-part of application No. 11/011,961, filed on Dec. 14, 2004, now abandoned.
Application No. 11/011,961 is a continuation of application No. 09/977,053, filed on Oct. 12, 2001, now abandoned.
(63) Continuation-in-part of application No. 10/859,018, filed on Jun. 1, 2004, now abandoned.
Application No. 10/859,018 is a continuation of application No. 10/038,288, filed on Nov. 9, 2001, now abandoned.
(63) Continuation-in-part of application No. 11/260,694, filed on Oct. 27, 2005, now abandoned.
Application No. 11/260,694 is a continuation of application No. 09/997,191, filed on Nov. 20, 2001, now abandoned.
(63) Continuation-in-part of application No. 11/039,397, filed on Jan. 20, 2005, now abandoned.
Application No. 11/039,397 is a continuation of application No. 10/154,675, filed on May 23, 2002, now abandoned.
(63) Continuation-in-part of application No. 11/149,003, filed on Jun. 9, 2005.
Application No. 11/149,003 is a continuation of application No. 10/189,971, filed on Jul. 3, 2002, now abandoned.
(63) Continuation-in-part of application No. 10/958,858, filed on Oct. 5, 2004, now abandoned.
Application No. 10/958,858 is a continuation of application No. 10/219,449, filed on Aug. 14, 2002, now abandoned.
(63) Continuation-in-part of application No. 11/022,296, filed on Dec. 23, 2004, now abandoned.
Application No. 11/022,296 is a continuation of application No. 10/843,131, filed on May 11, 2004, now Pat. No. 6,852,840.
Application No. 10/843,131 is a division of application No. 10/246,658, filed on Sep. 18, 2002, now Pat. No. 6,790,660.
(60) Provisional application No. 60/156,101, filed on Sep. 24, 1999.
(60) Provisional application No. 60/158,848, filed on Oct. 12, 1999.
(60) Provisional application No. 60/160,106, filed on Oct. 18, 1999.
(60) Provisional application No. 60/162,547, filed on Oct. 29, 1999.
(60) Provisional application No. 60/166,429, filed on Nov. 19, 1999.
(60) Provisional application No. 60/206,414, filed on May 23, 2000.
(60) Provisional application No. 60/216,384, filed on Jul. 7, 2000.
(60) Provisional application No. 60/219,890, filed on Jul. 21, 2000.
(60) Provisional application No. 60/230,609, filed on Sep. 6, 2000.
(60) Provisional application No. 60/218,461, filed on Jul. 14, 2000.
(60) Provisional application No. 60/232,283, filed on Sep. 13, 2000.
(60) Provisional application No. 60/232,793, filed on Sep. 15, 2000.
(60) Provisional application No. 60/234,100, filed on Sep. 21, 2000.
(60) Provisional application No. 60/235,744, filed on Sep. 27, 2000.
(60) Provisional application No. 60/241,195, filed on Oct. 17, 2000.
(60) Provisional application No. 60/240,466, filed on Oct. 13, 2000.
(60) Provisional application No. 60/249,044, filed on Nov. 15, 2000.
(60) Provisional application No. 60/252,361, filed on Nov. 21, 2000.
(60) Provisional application No. 60/293,709, filed on May 25, 2001.
(60) Provisional application No. 60/303,748, filed on Jul. 6, 2001.
(60) Provisional application No. 60/302,949, filed on Jul. 3, 2001.
(60) Provisional application No. 60/315,634, filed on Aug. 29, 2001.
(60) Provisional application No. 60/312,300, filed on Aug. 14, 2001.
(60) Provisional application No. 60/323,068, filed on Sep. 18, 2001.

(51) Int. Cl. C07H 21/04; C07K 16/00; C12N 15/00

(52) U.S. Cl. 435/320.1; 530/324; 536/23.5

(57) ABSTRACT

Novel human polynucleotide and polypeptide sequences are disclosed that can be used in therapeutic, diagnostic, and pharmacogenomic applications.

### 1.0 CROSS-REFERENCE TO RELATED APPLICATIONS

The present application is a continuation-in-part of: co-pending U.S. application Ser. No. 10/901,801, filed on Jul. 29, 2004, which is a continuation of U.S. application Ser. No. 09/667,380, filed on Sep. 22, 2000, abandoned, which claims the benefit of U.S. Provisional Application No. 60/156,101, filed on Sep. 24, 1999; co-pending U.S. application Ser. No. 09/689,911, filed on Oct. 11, 2000, which claims the benefit of U.S. Provisional Application No. 60/158,848, filed on Oct. 12, 1999; co-pending U.S. application Ser. No. 10/999,215, filed on Nov. 29, 2004, which is a continuation of U.S. application Ser. No. 09/691,343, filed on Oct. 18, 2000, abandoned, which claims the benefit of U.S. Provisional Application Nos. 60/162,547, filed on Oct. 29, 1999, and 60/160,106, filed on Oct. 18, 1999; co-pending U.S. application Ser. No. 11/285,738, filed on Nov. 22, 2005, which is a continuation of U.S. application Ser. No. 09/714,883, filed on Nov. 16, 2000, abandoned, which claims the benefit of U.S. Provisional Application No. 60/166,429, filed on Nov. 19, 1999; co-pending U.S. application Ser. No. 09/863,823, filed on May 23, 2001, which claims the benefit of U.S. Provisional Application No. 60/206,414, filed on May 23, 2000; co-pending U.S. application Ser. No. 11/039,362, filed on Jan. 19, 2005, which is a continuation of U.S. application Ser. No. 09/898,456, filed on Jul. 3, 2001, abandoned, which claims the benefit of U.S. Provisional Application Nos. 60/230,609, filed on Sep. 6, 2000, 60/219,890, filed on Jul. 21, 2000, and 60/216,384, filed on Jul. 7, 2000; co-pending U.S. application Ser. No. 09/899,514, filed on Jul. 5, 2001, which claims the benefit of U.S. Provisional Application No. 60/218,461, filed on Jul. 14, 2000; co-pending U.S. application Ser. No. 10/972,984, filed on Oct. 25, 2004, which is a continuation of U.S. application Ser. No. 09/952,474, filed on Sep. 12, 2001, abandoned, which claims the benefit of U.S. Provisional Application No. 60/232,283, filed on Sep. 13, 2000; co-pending U.S. application Ser. No. 11/049,637, filed on Feb. 2, 2005, which is a continuation of U.S. application Ser. No. 09/953,096, filed on Sep. 14, 2001, which issued as U.S. Pat. No. 6,867,291 B1 on Mar. 15, 2005, which claims the benefit of U.S. Provisional Application No. 60/232,793, filed on Sep. 15, 2000; co-pending U.S. application Ser. No. 11/012,588, filed on Dec. 15, 2004, which is a continuation of U.S. application Ser. No. 09/957,832, filed on Sep. 21, 2001, abandoned, which claims the benefit of U.S. Provisional Application No. 60/234,100, filed on Sep. 21, 2000; co-pending U.S. application Ser. No. 10/901,803, filed on Jul. 29, 2004, which is a continuation of U.S. application Ser. No. 09/962,740, filed on Sep. 25, 2001, abandoned, which claims the benefit of U.S. Provisional Application Nos. 60/241,195, filed on Oct. 17, 2000, and 60/235,744, filed on Sep. 27, 2000; co-pending U.S. application Ser. No. 11/011,961, filed on Dec. 14, 2004, which is a continuation of U.S. application Ser. No. 09/977,053, filed on Oct. 12, 2001, abandoned, which claims the benefit of U.S. Provisional Application No. 60/240,466, filed on Oct. 13, 2000; co-pending U.S. application Ser. No. 10/859,018, filed on Jun. 1, 2004, which is a continuation of U.S. application Ser. No. 10/038,288, filed on Nov. 9, 2001, abandoned, which claims the benefit of U.S. Provisional Application No. 60/249,044, filed on Nov. 15, 2000; co-pending U.S. application Ser. No. 11/260,694, filed on Oct. 27, 2005, which is a continuation of U.S. application Ser. No. 09/997,191, filed on Nov. 20, 2001, abandoned, which claims the benefit of U.S. Provisional Application No. 60/252,361, filed on Nov. 21, 2000; co-pending U.S. application Ser. No. 11/039,397, filed on Jan. 20, 2005, which is a continuation of U.S. application Ser. No. 10/154,675, filed on May 23, 2002, abandoned, which claims the benefit of U.S. Provisional Application Nos. 60/303,748, filed on Jul. 6, 2001, and 60/293,709, filed on May 23, 2001; co-pending U.S. application Ser. No. 11/149,003, filed on Jun. 9, 2005, which is a continuation of U.S. application Ser. No. 10/189,971, filed on Jul. 3, 2002, abandoned, which claims the benefit of U.S. Provisional Application Nos. 60/315,634, filed on Aug. 29, 2001, and 60/302,949, filed on Jul. 3, 2001; co-pending U.S. application Ser. No. 10/958,858, filed on Oct. 5, 2004, which is a continuation of U.S. application Ser. No. 10/219,449, filed on Aug. 14, 2002, abandoned, which claims the benefit of U.S. Provisional Application No. 60/312,300, filed on Aug. 14, 2001; and co-pending U.S. application Ser. No. 11/022,296, filed on Dec. 23, 2004, which is a continuation of U.S. application Ser. No. 10/843,131, filed on May 11, 2004, which issued as U.S. Pat. No. 6,852,840 B2 on Feb. 8, 2005, which is a divisional of U.S. application Ser. No. 10/246,658, filed on Sep. 18, 2002, which issued as U.S. Pat. No. 6,790,660 B1 on Sep. 14, 2004, which claims the benefit of U.S. Provisional Application No. 60/323,068, filed on Sep. 18, 2001; each of which is herein incorporated by reference in its entirety.

### 2.0 CROSS-REFERENCE TO SEQUENCE LISTING SUBMITTED ON COMPACT DISC

The present application contains a Sequence Listing of SEQ ID NOS:1-136, in file “FINALseqlist.TXT” (1,101,824 bytes), created on Feb. 10, 2006, submitted herewith on duplicate compact disc (Copy 1 and Copy 2), which is herein incorporated by reference in its entirety.

### 3.0 INTRODUCTION

The present invention relates to the discovery, identification, and characterization of novel human polynucleotides encoding proteins sharing sequence similarity with mammalian trypsin inhibitors, mammalian galanins, animal chordins, animal proteins that contain CUB domains, mammalian ceruloplasmins, animal proteins that contain an Ig-like domain, mammalian Wnt and Wnt-family proteins, mammalian cartilage matrix and von Willebrand factor proteins, mammalian netrin proteins, human hemicentin proteins, animal mucoid inhibitor proteins, mammalian cell adhesion proteins, human protein hormones, mammalian EGF-family proteins, animal collagen proteins, and animal kielin proteins. The invention encompasses the described polynucleotides, host cell expression systems, the encoded proteins, fusion proteins, polypeptides and peptides, antibodies to the encoded proteins and peptides, and genetically engineered animals that either lack or overexpress the disclosed polynucleotides, antagonists and agonists of the proteins, and other compounds that modulate the expression or activity of the proteins encoded by the disclosed polynucleotides, which can be used for diagnosis, drug screening, clinical trial monitoring, the treatment of physiological, behavioral, and/or infectious diseases and disorders, and cosmetic or nutriceutical applications.

### 4.0 BACKGROUND OF THE INVENTION

In addition to providing the structural and mechanical scaffolding for cells and tissues, proteins can also serve as recognition markers, ligands/receptors, mediate signal transduction and growth, mediate adhesion, and can mediate or facilitate the passage of materials across the lipid bilayer. Proteins are integral components of the various systems used by the body to monitor and regulate different bodily functions. Proteins present in the kidney and colon can mediate or modulate water resorption and blood volume in the body. In particular, secreted proteins, or circulating fragments or portions of other proteins, are often involved in regulating and maintaining a wide variety of biological and physiological processes. Often, such processes are mediated by protein ligands that interact with corresponding membrane receptor proteins that activate signal transduction and other pathways that control cell physiology, chemical release and communication, and gene expression.

Proteases are enzymes that mediate the proteolytic cleavage of polypeptide sequences. Conversely, protease inhibitors prevent or hinder proteolytic activity. Given the importance of proteolysis in a wide variety of cellular functions and disease, protease inhibitors have been demonstrated to be involved in, inter alia, regulating development, modulating cellular processes, and preventing infectious, and particularly viral, disease.

Galanins are biologically active peptides that are present in the central and peripheral nervous system and are upregulated after spinal injury and in response to estrogen. Galanins also include neuropeptides that control a broad variety of biological activities such as, for example, the release of growth hormone, inhibition of insulin and somatostatin release, smooth muscle contraction in the gastrointestinal and genitourinary tracts, and adrenal secretion. Galanins are typically cleaved from longer precursor proteins and are about 29-30 amino acids in length. The first 14 residues of mature galanin proteins are highly conserved. Galanins have been associated with, inter alia, regulating body weight, modulating behavior, treating pain, inflammation, neuronal repair, Alzheimer's dementia, inflammatory bowel disorders, and infectious disease.

Ceruloplasmins are members of a family of metal chelating proteins. Ceruloplasmins have been associated with development, ferroxidase activity, amine oxidase activity, copper transport, homeostasis, and superoxide dismutase activity. Wnt and Wnt-family proteins are soluble secreted growth and signaling proteins that have been implicated in a number of biological processes and anomalies, such as blood cell formation, cancer, homeostasis, development (i.e., intercellular signaling during vertebrate (especially spinal cord) development), weight regulation, and inflammation.

Von Willebrand proteins are secreted proteins that have been implicated in cartilage formation and development and platelet binding to circulatory endothelium. Netrins are secreted proteins that have been implicated in a number of biological processes and anomalies such as neural development, paralysis, and axon guidance. Kielins are secreted proteins that have been implicated in a number of biological processes and anomalies such as development and signal transduction. Collagens are a family of proteins that are among the most abundant proteins in the body. Biosynthetically produced collagens find medical utility in prosthetic and cosmetic applications.

Therefore, secreted proteins constitute ideal targets for drug intervention and for the design of therapeutic agents.

### 5.0 SUMMARY OF THE INVENTION

The present invention relates to the discovery, identification, and characterization of nucleotides that encode novel human secreted proteins, and the corresponding amino acid sequences of these proteins. The novel human secreted proteins, described for the first time herein, share structural similarity with: animal trypsin inhibitor proteins, cancer pathogenesis proteins, sperm glycoproteins, and secretory proteins (SEQ ID NOS:1-3); animal galanins (SEQ ID NOS:4-7; unlike other known galanins, the presently described sequences differ at amino acid 14 of the consensus sequence shared by other galanins, replacing a histidine residue in the consensus with a valine residue at position 46 of SEQ ID NOS:5 and 7); animal chordins, NEL protein, and thrombospondin (SEQ ID NOS:8-12); animal proteins that contain CUB domains (SEQ ID NOS:13 and 14); animal ceruloplasmins (SEQ ID NOS:15 and 16); eukaryotic membrane and secreted proteins, including, but not limited to, neural cell adhesion molecules (NCAMs), via the Ig-like domain, tyrosine kinase receptors, and vascular endothelial growth factor (VEGF) receptors (SEQ ID NOS:17-25); animal Wnt proteins, particularly Wnt-3A (SEQ ID NOS:26-30) and Wnt-8D (SEQ ID NOS:31-49); animal cartilage matrix proteins and von Willebrand proteins (SEQ ID NOS:50-52); animal netrin, laminin, agrin, and attractin proteins (SEQ ID NOS:53-57); mammalian hemicentin, titin, basement membrane, semaphorin, fibulin, and cell adhesion proteins (SEQ ID NOS:58-61); animal protease inhibitors, serine protease inhibitors, follistatin, and ovomucoid inhibitors (SEQ ID NOS:62-66); animal protease inhibitors, antithrombin, serine protease inhibitors, plasminogen activator inhibitor, serpins, neurite promoting-factor, and nexins (SEQ ID NOS:67-71); mammalian cell adhesion proteins, selectins, and a variety of cell surface markers and receptors (SEQ ID NOS:72-78); animal Wnt-family proteins, disintegrins, and metalloproteinases (SEQ ID NOS:79-83); human protein hormones chorionic gonadotrophin and follicle stimulating hormone (SEQ ID NOS:84-86); animal Wnt-family proteins, in particular the human ortholog of chicken Wnt-14 (SEQ ID NOS:87-89); mammalian proteins of the epidermal growth factor (EGF) superfamily and notch proteins (SEQ ID NOS:90-103); animal kielin and chordin proteins (SEQ ID NOS:104-128); animal collagens, including, but not limited to, the human collagen alpha 2 (VIII) chain (SEQ ID NOS:129-132); and animal kielin, zonadhesin, and chordin proteins (note the high cysteine content) (SEQ ID NOS:133-136).

Galanins are typically produced as longer precursor proteins that are subsequently cleaved (at one or both ends) into their mature or active form. The galanin-like consensus sequence begins at amino acid number 33 of SEQ ID NOS:5 and 7, and this position will generally define the amino terminus of the mature form of the disclosed galanin-like sequences. Galanins are typically about 29-30 amino acids in length. Accordingly, an additional aspect of the present invention includes peptides having an N-terminus beginning at amino acid position 33 of SEQ ID NOS:5 or 7, extending at least about 14 amino acids in length, and having a carboxy-terminus at any amino acid position disclosed in the Sequence Listing, and the polynucleotide sequences encoding the same.

As neuropeptides, galanins have been subject to intense scientific scrutiny. For examples of how the described galanin-like proteins, or their (G-protein coupled) receptors, can be produced, antagonized, processed, applied, and delivered, see, for example, U.S. Pat. Nos. 5,576,296 and 5,756,460, U.S. Provisional Patent Application Ser. No. 60/033,851, and U.S. patent application Ser. No. 08/721,837. Given their structural relatedness to galanins, the described galanin-like sequences are suitable for use and modification as contemplated for other galanins.

With regard to SEQ ID NOS:8-14, upon secretion these proteins typically exert physiological effect by interacting with receptors to produce a biological effect (such as, for example, signal transduction). Consequently, interfering with the binding of these proteins to their cognate receptors effect processes mediated by these proteins, while enhancing the concentration of these proteins in vivo can boost the effects/activity levels of such processes. Yet another alternative is that these proteins, or portions thereof, can act as hormones (or peptide hormones), enzymes, or receptor/ligand antagonists, and used accordingly. As such, these proteins have been the subject of intense scientific and commercial scrutiny (see, e.g., PCT Patent Application Serial Nos. PCT/US98/04858, filed Mar. 12, 1998, and PCT/US98/05255, filed Mar. 18, 1998, U.S. Patent Application Serial No. 09/040,963, filed Mar. 18, 1998, and U.S. Provisional Patent Application Nos. 60/068,368, filed Dec. 19, 1997, 60/057,765, filed Sep. 5, 1997, 60/048,970, filed Jun. 6, 1997, 60/040,762, filed Mar. 14, 1997, and 60/041,263, filed Mar. 19, 1997.

With respect to SEQ ID NOS:8-12, chordins are developmentally active proteins that are antagonists of bone morphogenic protein-4 (BMP-4), and serve as targets for proteolytic cleavage by BMP-1. Chordin has been implicated in developmental regulation during gastrulation and skeletogenesis. The regions of SEQ ID NOS:9 and 11 that constitute the chordin-like domains also display marked similarity with human NEL protein and animal thrombospondins. In addition to development, these proteins have been associated with biological activities such as, for example, the inhibition of angiogenesis, clotting, and adrenal secretion.

With respect to SEQ ID NOS:13 and 14, the CUB domain is an extracellular domain (ECD) present in variety of diverse proteins, such as BMP-1, proteinases, spermadhesins, complement subcomponents, and neuronal recognition molecules. SEQ ID NO:14 also displays significant similarity with bone morphogenic protein, neuropilin, C-proteinases and endopeptidases, human NP-2, semaphorin, bovine acidic seminal fluid protein, and vascular endothelial growth factor. Thus, SEQ ID NO:14 represents a new member of the platelet-derived growth factor/VEGF family of proteins.

With respect to SEQ ID NOS:15 and 16, as ceruloplasmins are metal chelating proteins involved in copper transport, ceruloplasmins have been implicated in conditions including, but not limited to, Wilson's Disease.

As secreted growth factors, Wnt-family proteins have been subject to considerable scrutiny, as evidenced by U.S. Pat. Nos. 5,824,789, 6,043,053, and 5,780,291, which describe a variety of assays and applications that are applicable to the presently described Wnt-family proteins.

SEQ ID NOS:90-103 can be used in drug screening assays similar to those described in, for example, U.S. Pat. No. 6,048,850, in order to identify compounds for treating diseases such as, for example, immune disorders, Alzheimer's disease, epilepsy, and Parkinson's disease.

Given the physiological importance of collagen proteins, they have been subject to intense scrutiny as exemplified and discussed in U.S. Pat. Nos. 5,925,736 and 5,807,581, which describe a variety of uses and applications applicable to the presently described collagen proteins.

The novel human nucleic acid sequence described herein encode alternative proteins/open reading frames (ORFs) of 497, 141, 116, 451, 429, 305, 996, 254, 210, 262, 218, 423, 352, 369, 351, 255, 34, 23, 36, 351, 34, 36, 449, 288, 261, 5518, 4126, 86, 70, 404, 362, 1107, 3571, 1842, 433, 363, 84, 365, 995, 1130, 709, 844, 790, 925, 955, 1628, 1593, 1057, 1477, 1512, 1570, 1535, 1251, 1192, 1207, 759, 1342, 717, 703, 685, and 627 amino acids in length (see SEQ ID NOS:2, 5, 7, 9, 11, 14, 16, 18, 20, 22, 24, 27, 29, 32, 34, 36, 38, 40, 42, 44, 46, 48, 51, 54, 56, 59, 61, 63, 65, 68, 70, 73, 75, 77, 80, 82, 85, 88, 91, 93, 95, 97, 99, 101, 103, 105, 107, 109, 111, 113, 115, 117, 119, 121, 123, 125, 127, 130, 132, 134, and 136, respectively). SEQ ID NOS:3, 12, 25, 30, 49, 52, 57, 66, 71, 78, 83, 86, 89, and 128 describe full length ORFs, as well as flanking 5′ and 3′ sequences.

The invention also encompasses agonists and antagonists of the described secreted proteins, including small molecules, large molecules, mutant versions of the described secreted proteins, or portions thereof, that compete with native secreted proteins, peptides, antibodies, nucleotide sequences that can be used to inhibit (e.g., antisense and ribozyme molecules, and open reading frame or regulatory sequence replacement constructs) or enhance (e.g., expression constructs that place the described polynucleotides under the control of a strong promoter system) the expression of the described secreted proteins, and transgenic animals that express the described secreted protein sequences, or “knock-outs” (which can be conditional) that do not express functional versions of the described secreted proteins. Knock-out mice can be produced in several ways, one of which involves the use of mouse embryonic stem cell lines that contain gene trap mutations in a murine homolog of at least one of the described secreted protein sequences. When the unique secreted protein sequences described in SEQ ID NOS:1-136 are “knocked-out” they provide a method of identifying phenotypic expression of the particular gene, as well as a method of assigning function to previously unknown genes. In addition, animals in which the unique secreted protein sequences described in SEQ ID NOS:1-136 are “knocked-out” provide an unique source in which to elicit antibodies to homologous and orthologous proteins, which would have been previously viewed by the immune system as “self” and therefore would have failed to elicit significant antibody responses.

Additionally, the unique secreted protein sequences described in SEQ ID NOS:1-136 are useful for the identification of protein coding sequences, and mapping an unique gene to a particular chromosome. These sequences identify biologically verified exon splice junctions, as opposed to splice junctions that may have been bioinformatically predicted from genomic sequence alone. The sequences of the present invention are also useful as additional DNA markers for restriction fragment length polymorphism (RFLP) analysis, and in forensic biology, particularly given the presence of nucleotide polymorphisms within the described sequences.

Further, the present invention also relates to processes for identifying compounds that modulate, i.e., act as agonists or antagonists of, expression and/or activity of the described secreted protein sequences that utilize purified preparations of the described secreted protein nucleotide and/or polypeptide products, or cells expressing the same. Such compounds can be used as therapeutic agents for the treatment of any of a wide variety of symptoms associated with biological disorders or imbalances.

### 6.0 BRIEF DESCRIPTION OF THE FIGURES

No Figures are required in the present invention.

### 7.0 DETAILED DESCRIPTION OF THE INVENTION

The human secreted proteins described for the first time herein are novel proteins that are apparently expressed in, inter alia, human cell lines and: human prostate, fetal brain, cerebellum, spinal cord, thymus, spleen, lymph node, bone marrow, trachea, lung, kidney, fetal liver, thyroid, adrenal gland, stomach, small intestine, colon, muscle, heart, uterus, placenta, mammary gland, and testis cells (SEQ ID NOS:1-3); human cervix cells (SEQ ID NOS:8-14); human testis and mammary gland cells (SEQ ID NOS:15 and 16); human kidney, colon, and rectum cells (SEQ ID NOS:17-25); human adipose, esophagus, cervix, prostate, testis, and pericardium cells (SEQ ID NOS:26-30); human pituitary gland, cerebellum, spleen, adrenal gland, small intestine, skeletal muscle, heart, uterus, adipose, esophagus, cervix, rectum, pericardium, fetal kidney, and fetal lung cells (SEQ ID NOS:31-49); human uterus, adipose, esophagus, cervix, brain, prostate, trachea, thyroid, spleen, and rectum cells (SEQ ID NOS:50-52); human lymph node, testis, heart, mammary gland, adipose, esophagus, cervix, pericardium, fetal kidney, fetal lung, 6-, 9-, and 12-wk embryo, brain, pituitary, spleen, activated T cells, skeletal muscle, and fetal brain cells (SEQ ID NOS:53-57); human fetal brain, spinal cord, thymus, pituitary, lymph node, trachea, kidney, liver, prostate, testis, stomach, small intestine, skeletal muscle, adrenal gland, heart, uterus, mammary gland, adipose, skin, esophagus, bladder, cervix, rectum, pericardium, and ovary cells (SEQ ID NOS:58-61); human thymus and testis cells (SEQ ID NOS:62-66); human fetal brain, spinal cord, spleen, testis, and adipose cells (SEQ ID NOS:67-71); human cerebellum, pituitary gland, bone marrow, testis, adrenal gland, small intestine, heart, uterus, placenta, mammary gland, adipose, esophagus, cervix, rectum, pericardium, fetal kidney, and fetal lung cells (SEQ ID NOS:72-78); human brain, pituitary, cerebellum, thymus, spleen, lymph node, kidney, fetal liver, prostate, testis, thyroid, adrenal gland, salivary gland, stomach, small intestine, colon, skeletal muscle, heart, uterus, placenta, mammary gland, adipose, esophagus, bladder, cervix, rectum, pericardium, hypothalamus, ovary, fetal kidney, and fetal lung cells (SEQ ID NOS:79-83); human fetal brain, spinal cord, thymus, lymph node, lung, kidney, testis, adrenal gland, bone marrow, stomach, small intestine, colon, uterus, placenta, mammary gland, bladder, hypothalamus, fetal kidney, fetal lung, gall bladder, aorta, osteosarcoma, 6-, 9-, and 12-week embryo, embryonic carcinoma, and microvascular endothelium cells (SEQ ID NOS:84-86); human fetal tissue and testis cells (SEQ ID NOS:87-89); human brain, hypothalamus, lymph node, fetal kidney, fetal lung, and 6- and 9-week old embryo cells (SEQ ID NOS:90-101); human liver, spleen, pituitary, lymph node, fetal kidney, and fetal lung cells (SEQ ID NOS:102-103); human brain, bone marrow, adrenal gland, liver, lymph node, mammary gland, prostate, pancreas, pituitary, placenta, thymus, trachea, skeletal muscle, kidney, thyroid, testis, activated T-cells, spleen, fetal brain, lung, umbilical vein endothelium, and fetal kidney cells (SEQ ID NOS:104-128); human pituitary, lymph node, fetal kidney, and osteocarcinoma cells (SEQ ID NOS:129-132); and fetal brain, brain, pituitary, cerebellum, spinal cord, thymus, spleen, lymph node, bone marrow, trachea, lung, kidney, fetal liver, liver, prostate, testis, thyroid, adrenal gland, pancreas, salivary gland, stomach, small intestine, colon, skeletal muscle, heart, uterus, placenta, mammary gland, adipose, skin, esophagus, bladder, cervix, rectum, pericardium, eye, ovary, fetal kidney, fetal lung, gall bladder, tongue, aorta, 6-, 9-, and 12-week old embryos, osteosarcoma, embryonic carcinoma, umbilical vein, and microvascular endothelial cells (SEQ ID NOS:133-136).

The present invention encompasses the nucleotides presented in the Sequence Listing, host cells expressing such nucleotides, the expression products of such nucleotides, and: (a) nucleotides that encode mammalian homologs of the described nucleotides, including the specifically described secreted protein nucleotide sequences, and related secreted protein products; (b) nucleotides that encode one or more portions of the described secreted proteins corresponding to a secreted protein functional domain(s), and the polypeptide products specified by such nucleotide sequences, including, but not limited to, the novel regions of any active domain(s); (c) isolated nucleotides that encode mutant versions, engineered or naturally occurring, of the described secreted proteins, in which all or a part of at least one domain is deleted or altered, and the polypeptide products specified by such nucleotide sequences, including, but not limited to, soluble proteins and peptides; (d) nucleotides that encode chimeric fusion proteins containing all or a portion of a coding region of a secreted protein, or one of its domains (e.g., a receptor or ligand binding domain, accessory protein/self-association domain, etc.) fused to another peptide or polypeptide; or (e) therapeutic or diagnostic derivatives of the described polynucleotides, such as oligonucleotides, antisense polynucleotides, ribozymes, dsRNA, or gene therapy constructs, comprising a sequence first disclosed in the Sequence Listing.

As discussed above, the present invention includes the human DNA sequences presented in the Sequence Listing (and vectors comprising the same), and additionally contemplates any nucleotide sequence encoding a contiguous secreted protein open reading frame (ORF) that hybridizes to a complement of a DNA sequence presented in the Sequence Listing under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (“Current Protocols in Molecular Biology”, Vol. 1, p. 2.10.3 (Ausubel et al., eds., Green Publishing Associates, Inc., and John Wiley & Sons, Inc., New York, 1989)) and encodes a functionally equivalent expression product. Additionally contemplated are any nucleotide sequences that hybridize to the complement of a DNA sequence that encodes and expresses an amino acid sequence presented in the Sequence Listing under moderately stringent conditions, e.g., washing in 0.2×SSC/0.1% SDS at 42° C. (“Current Protocols in Molecular Biology”, supra), yet still encode a functionally equivalent secreted protein product. Functional equivalents of the described secreted proteins include naturally occurring homologs of the described secreted proteins present in other species, and mutant versions of the described secreted proteins, whether naturally occurring or engineered (by site directed mutagenesis, gene shuffling, directed evolution as described in, for example, U.S. Pat. No. 5,837,458). The invention also includes degenerate nucleic acid variants of the disclosed secreted protein polynucleotide sequences.

Additionally contemplated are polynucleotides encoding secreted protein ORFs, or their functional equivalents, encoded by polynucleotide sequences that are about 99, 95, 90, or about 85 percent similar or identical to corresponding regions of the nucleotide sequences of the Sequence Listing (as measured by BLAST sequence comparison analysis using, for example, the GCG sequence analysis package (the University of Wisconsin GCG sequence analysis package, SEQUENCHER 3.0, Gene Codes Corp., Ann Arbor, Mich.) using default settings).

The invention also includes nucleic acid molecules, preferably DNA molecules, that hybridize to, and are therefore the complements of, the described secreted protein nucleotide sequences. Such hybridization conditions may be highly stringent or less highly stringent, as described herein. In instances where the nucleic acid molecules are deoxyoligonucleotides, such molecules are generally about 16 to about 100 bases long, or about 20 to about 80 bases long, or about 34 to about 45 bases long, or any variation or combination of sizes represented therein that incorporate a contiguous region of sequence first disclosed in the Sequence Listing. Such oligonucleotides can be used in conjunction with the polymerase chain reaction (PCR) to screen libraries, isolate clones, and prepare cloning and sequencing templates, etc.

Alternatively, such secreted protein oligonucleotides can be used as hybridization probes for screening libraries, and assessing gene expression patterns (particularly using a microarray or high-throughput “chip” format). Additionally, a series of oligonucleotide sequences, or the complements thereof, can be used to represent all or a portion of the described secreted protein sequences. An oligonucleotide or polynucleotide sequence first disclosed in at least a portion of one or more of the sequences of SEQ ID NOS:1-136 can be used as a hybridization probe in conjunction with a solid support matrix/substrate (resins, beads, membranes, plastics, polymers, metal or metallized substrates, crystalline or polycrystalline substrates, etc.). Of particular note are spatially addressable arrays (i.e., gene chips, microtiter plates, etc.) of oligonucleotides and polynucleotides, or corresponding oligopeptides and polypeptides, wherein at least one of the biopolymers present on the spatially addressable array comprises an oligonucleotide or polynucleotide sequence first disclosed in at least one of the sequences of SEQ ID NOS:1-136, or an amino acid sequence encoded thereby. Methods for attaching biopolymers to, or synthesizing biopolymers on, solid support matrices, and conducting binding studies thereon, are disclosed in, inter alia, U.S. Pat. Nos. 5,700,637, 5,556,752, 5,744,305, 4,631,211, 5,445,934, 5,252,743, 4,713,326, 5,424,186, and 4,689,405.

Addressable arrays comprising sequences first disclosed in SEQ ID NOS:1-136 can be used to identify and characterize the temporal and tissue specific expression of a gene. These addressable arrays incorporate oligonucleotide sequences of sufficient length to confer the required specificity, yet be within the limitations of the production technology. The length of these probes is usually within a range of between about 8 to about 2000 nucleotides. Preferably the probes consist of 60 nucleotides, and more preferably 25 nucleotides, from the sequences first disclosed in SEQ ID NOS:1-136.

For example, a series of oligonucleotide sequences, or the complements thereof, can be used in chip format to represent all or a portion of the described secreted protein sequences. The oligonucleotides, typically between about 16 to about 40 (or any whole number within the stated range) nucleotides in length, can partially overlap each other, and/or the sequence may be represented using oligonucleotides that do not overlap. Accordingly, the described polynucleotide sequences shall typically comprise at least about two or three distinct oligonucleotide sequences of at least about 8 nucleotides in length that are each first disclosed in the described Sequence Listing. Such oligonucleotide sequences can begin at any nucleotide present within a sequence in the Sequence Listing, and proceed in either a sense (5′-to-3′) orientation vis-a-vis the described sequence or in an antisense orientation.

Microarray-based analysis allows the discovery of broad patterns of genetic activity, providing new understanding of gene functions, and generating novel and unexpected insight into transcriptional processes and biological mechanisms. The use of addressable arrays comprising sequences first disclosed in SEQ ID NOS:1-136 provides detailed information about transcriptional changes involved in a specific pathway, potentially leading to the identification of novel components, or gene functions that manifest themselves as novel phenotypes.

Probes consisting of sequences first disclosed in SEQ ID NOS:1-136 can also be used in the identification, selection, and validation of novel molecular targets for drug discovery. The use of these unique sequences permits the direct confirmation of drug targets, and recognition of drug dependent changes in gene expression that are modulated through pathways distinct from the intended target of the drug. These unique sequences therefore also have utility in defining and monitoring both drug action and toxicity.

As an example of utility, the sequences first disclosed in SEQ ID NOS:1-136 can be utilized in microarrays, or other assay formats, to screen collections of genetic material from patients who have a particular medical condition. These investigations can also be carried out using the sequences first disclosed in SEQ ID NOS:1-136 in silico, and by comparing previously collected genetic databases and the disclosed sequences using computer software known to those in the art.

Thus the sequences first disclosed in SEQ ID NOS:1-136 can be used to identify mutations associated with a particular disease, and also in diagnostic or prognostic assays.

Although the presently described sequences have been specifically described using nucleotide sequence, it should be appreciated that each of the sequences can uniquely be described using any of a wide variety of additional structural attributes, or combinations thereof. For example, a given sequence can be described by the net composition of the nucleotides present within a given region of the sequence, in conjunction with the presence of one or more specific oligonucleotide sequence(s) first disclosed in SEQ ID NOS:1-136. Alternatively, a restriction map specifying the relative positions of restriction endonuclease digestion sites, or various palindromic or other specific oligonucleotide sequences, can be used to structurally describe a given sequence. Such restriction maps, which are typically generated by widely available computer programs (e.g., the University of Wisconsin GCG sequence analysis package, SEQUENCHER 3.0, Gene Codes Corp., etc.), can optionally be used in conjunction with one or more discrete nucleotide sequence(s) present in the sequence that can be described by the relative position of the sequence relative to one or more additional sequence(s) or one or more restriction sites present in the disclosed sequence.

For oligonucleotide probes, highly stringent conditions may refer, e.g., to washing in 6×SSC/0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides). These nucleic acid molecules may encode or act as antisense molecules, useful, for example, in gene regulation of the described secreted protein nucleic acid sequences and/or as antisense primers in amplification reactions of the described secreted protein nucleic acid sequences. With respect to gene regulation, such techniques can be used to regulate biological functions. Further, such sequences may be used as part of ribozyme and/or triple helix sequences that are also useful for gene regulation of the described secreted protein nucleic acid sequences.

Inhibitory antisense or double stranded oligonucleotides can additionally comprise at least one modified base moiety that is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, and 2,6-diaminopurine.

The antisense oligonucleotide can also comprise at least one modified sugar moiety selected from the group including, but not limited to, arabinose, 2-fluoroarabinose, xylulose, and hexose.

In yet another embodiment, the antisense oligonucleotide will comprise at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-6641, 1987). The oligonucleotide is a 2′-0-methylribonucleotide (Inoue et al., Nucl. Acids Res. 15:6131-6148, 1987), or a chimeric RNA-DNA analogue (Inoue et al., FEBS Lett. 215:327-330, 1987). Alternatively, double stranded RNA can be used to disrupt the expression and function of a targeted secreted protein sequence.

Oligonucleotides of the invention can be synthesized by standard methods known in the art, e.g., by use of an automated DNA synthesizer (such as are commercially available from Biosearch Technologies, Inc., Novato, Calif., Applied Biosystems, Foster City, Calif., etc.). As examples, phosphorothioate oligonucleotides can be synthesized (Stein et al., Nucl. Acids Res. 16:3209-3221, 1988), and methylphosphonate oligonucleotides can be prepared by use of controlled pore glass polymer supports (Sarin et al., Proc. Natl. Acad. Sci. USA 85:7448-7451, 1988), etc.

Low stringency conditions are well-known to those of skill in the art, and will vary predictably depending on the specific organisms from which the library and the labeled sequences are derived. For guidance regarding such conditions, see, for example, “Molecular Cloning, A Laboratory Manual” (Sambrook et al., eds., Cold Spring Harbor Press, Cold Spring Harbor, N.Y., 1989), “Current Protocols in Molecular Biology”, supra, and periodic updates thereof.

Alternatively, suitably labeled secreted protein nucleotide probes can be used to screen a human genomic library using appropriately stringent conditions or by PCR. The identification and characterization of human genomic clones is helpful for identifying polymorphisms (including, but not limited to, nucleotide repeats, microsatellite alleles, single nucleotide polymorphisms, or coding single nucleotide polymorphisms), determining the genomic structure of a given locus/allele, and designing diagnostic tests. For example, sequences derived from regions adjacent to the intron/exon boundaries of the human gene can be used to design primers for use in amplification assays to detect mutations within the exons, introns, splice sites (e.g., splice acceptor and/or donor sites), etc., that can be used in diagnostics and pharmacogenomics.

For example, the present sequences can be used in restriction fragment length polymorphism (RFLP) analysis to identify specific individuals. In this technique, an individual's genomic DNA is digested with one or more restriction enzymes, and probed on a Southern blot to yield unique bands for identification (as generally described in U.S. Pat. No. 5,272,057). In addition, the sequences of the present invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). Actual base sequence information can be used for identification as an accurate alternative to patterns formed by restriction enzyme generated fragments.

Further, homologs of the described secreted protein sequences can be isolated from nucleic acid from an organism of interest by performing PCR using two degenerate or “wobble” oligonucleotide primer pools designed on the basis of amino acid sequences within the secreted protein products disclosed herein. The template for the reaction may be genomic DNA, or total RNA, mRNA, and/or cDNA obtained by reverse transcription of mRNA, prepared from human or non-human cell lines or tissue known to express, or suspected of expressing, an allele of a gene encoding the described secreted proteins. The PCR product can be subcloned and sequenced to ensure that the amplified sequences represent the sequence of the desired secreted protein gene. The PCR fragment can then be used to isolate a full length cDNA clone by a variety of methods. For example, the amplified fragment can be labeled and used to screen a cDNA library, such as a bacteriophage cDNA library. Alternatively, the labeled fragment can be used to isolate genomic clones via the screening of a genomic library.

PCR technology can also be used to isolate full length cDNA sequences. For example, RNA can be isolated, following standard procedures, from an appropriate cellular or tissue source (i.e., one known to express, or suspected of expressing, a gene encoding the described secreted proteins). A reverse transcription (RT) reaction can be performed on the RNA using an oligonucleotide primer specific for the most 5′ end of the amplified fragment for the priming of first strand synthesis. The resulting RNA/DNA hybrid may then be “tailed” using a standard terminal transferase reaction, the hybrid may be digested with RNase H, and second strand synthesis may then be primed with a complementary primer. Thus, cDNA sequences upstream of the amplified fragment can be isolated. For a review of cloning strategies that can be used, see, e.g., “Molecular Cloning, A Laboratory Manual”, supra.

A cDNA encoding a mutant version of the described secreted protein sequences can be isolated, for example, by using PCR. In this case, the first cDNA strand may be synthesized by hybridizing an oligo-dT oligonucleotide to mRNA isolated from tissue known to express, or suspected of expressing, the described secreted proteins, in an individual putatively carrying a mutant allele of a gene encoding the described secreted proteins, and by extending the new strand with reverse transcriptase. The second strand of the cDNA is then synthesized using an oligonucleotide that hybridizes specifically to the 5′ end of the normal sequence. Using these two primers, the product is then amplified via PCR, optionally cloned into a suitable vector, and subjected to DNA sequence analysis through methods well-known to those of skill in the art. By comparing the DNA sequence of the mutant allele to that of a corresponding normal allele, the mutation(s) responsible for the loss or alteration of function of the mutant version of the described secreted protein gene products can be ascertained.

Alternatively, a genomic library can be constructed using DNA obtained from an individual suspected of carrying, or known to carry, a mutant allele of a gene encoding the described secreted proteins (e.g., a person manifesting a phenotype associated with the described secreted proteins, such as, for example, abnormal body weight, obesity, cardiovascular disease, hyperproliferative disorders, high blood pressure, thrombosis, restenosis, disorders of the joints or circulatory systems, abnormal blood clotting, cancer, developmental defects, paralysis or palsy, nerve damage or degeneration, osteoporosis, connective tissue disorders, infertility, an inflammatory disorder, arthritis, Wilson's disease, vision disorders, etc.), or a cDNA library can be constructed using RNA from a tissue known to express, or suspected of expressing, a mutant allele of a gene encoding the described secreted proteins. A normal allele of a gene encoding the described secreted proteins, or any suitable fragment thereof, can then be labeled and used as a probe to identify the corresponding mutant allele of a gene encoding the described secreted proteins in such libraries. Clones containing mutant versions of the described secreted proteins can then be purified and subjected to sequence analysis according to methods well-known to those skilled in the art.

Additionally, an expression library can be constructed utilizing cDNA synthesized from, for example, RNA isolated from a tissue known to express, or suspected of expressing, a mutant allele of a gene encoding the described secreted proteins, in an individual suspected of carrying, or known to carry, such a mutant allele. In this manner, gene products made by the putatively mutant tissue can be expressed and screened using standard antibody screening techniques in conjunction with antibodies raised against a normal version of the described secreted protein product, as described below (for screening techniques, see, for example, “Antibodies: A Laboratory Manual” (Harlow and Lane, eds., Cold Spring Harbor Press, Cold Spring Harbor, N.Y., 1988)).

Additionally, screening can be accomplished by screening with labeled secreted protein fusion proteins, such as, for example, alkaline phosphatase-secreted protein or secreted protein-alkaline phosphatase fusion proteins. In cases where a mutation of the described secreted proteins results in an expression product with altered function (e.g., as a result of a missense or a frameshift mutation), polyclonal antibodies to the described secreted proteins are likely to cross-react with a corresponding mutant version of the described secreted proteins. Library clones detected via their reaction with such labeled antibodies can be purified and subjected to sequence analysis according to methods well-known in the art.

The invention also encompasses: (a) DNA vectors that contain any of the foregoing secreted protein coding sequences and/or their complements (i.e., antisense); (b) DNA expression vectors that contain any of the foregoing secreted protein coding sequences operatively associated with a regulatory element that directs the expression of the coding sequences (for example, baculovirus as described in U.S. Pat. No. 5,869,336 herein incorporated by reference); (c) genetically engineered host cells that contain any of the foregoing secreted protein coding sequences operatively associated with a regulatory element that directs the expression of the coding sequences in the host cell; and (d) genetically engineered host cells that express an endogenous secreted protein sequence under the control of an exogenously introduced regulatory element (i.e., gene activation). As used herein, regulatory elements include, but are not limited to, inducible and non-inducible promoters, enhancers, operators, and other elements known to those skilled in the art that drive and regulate expression. Such regulatory elements include, but are not limited to, the cytomegalovirus (hCMV) immediate early gene, regulatable, viral elements (particularly retroviral LTR promoters), the early or late promoters of SV40 or adenovirus, the lac system, the trp system, the TAC system, the TRC system, the major operator and promoter regions of phage lambda, the control regions of fd coat protein, the promoter for 3-phosphoglycerate kinase (PGK), the promoters of acid phosphatase, and the promoters of the yeast α-mating factors.

The present invention also encompasses antibodies and anti-idiotypic antibodies (including Fab fragments), antagonists and agonists of the described secreted proteins, as well as compounds or nucleotide constructs that inhibit (transcription factor inhibitors, antisense and ribozyme molecules, or open reading frame sequence or regulatory sequence replacement constructs), or promote (e.g., expression constructs in which secreted protein coding sequences are operatively associated with expression control elements, such as promoters, promoter/enhancers, etc.) expression of the described secreted proteins.

The described secreted proteins, peptides, fusion proteins, nucleotide sequences, antibodies, antagonists, and agonists can be useful for the detection of mutant or inappropriately expressed versions of the described secreted proteins for the diagnosis of disease. The described secreted proteins, peptides, fusion proteins, nucleotide sequences, host cell expression systems, antibodies, antagonists, agonists, and genetically engineered cells and animals can be used for screening for drugs (or high throughput screening of combinatorial libraries) effective in the treatment of the symptomatic or phenotypic manifestations of perturbing the normal function of the described secreted proteins in the body. The use of engineered host cells and/or animals may offer an advantage in that such systems allow not only for the identification of compounds that bind to an endogenous receptor for the described secreted proteins, but can also identify compounds that trigger activities or pathways mediated by the described secreted proteins.

Finally, the described secreted protein products can be used as therapeutics (i.e., for the treatment of Wilson's Disease, etc.). For example, soluble derivatives, such as a mature version of the described secreted proteins, peptides or domains corresponding to the described secreted proteins, secreted protein fusion protein products (especially Ig fusion proteins, i.e., fusions of the described secreted proteins, or a domain of the described secreted proteins, to an IgFc), secreted protein antibodies and anti-idiotypic antibodies (including Fab fragments), antagonists or agonists (including compounds that modulate or act on downstream targets in a pathway mediated by the described secreted proteins) can be used to directly treat diseases or disorders. For instance, the administration of an effective amount of a soluble secreted protein, a secreted protein-IgFc fusion protein, or an anti-idiotypic antibody (or its Fab) that mimics the secreted protein, could activate or effectively antagonize an endogenous secreted protein receptor. Soluble versions of the described secreted proteins can also be modified by proteolytic cleavage to active peptide products (e.g., any novel peptide sequence initiating at any one of the amino acids presented in the Sequence Listing and ending at any downstream amino acid). Such products or peptides can be further subject to modification such as the construction of secreted protein fusion proteins and/or can be derivatized by being combined with pharmaceutically acceptable agents such as, but not limited to, polyethylene glycol (PEG).

Nucleotide constructs encoding such secreted protein products can be used to genetically engineer host cells to express such products in vivo; these genetically engineered cells function as “bioreactors” in the body delivering a continuous supply of the described secreted proteins, peptides, or fusion proteins to the body. Nucleotide constructs encoding functional or mutant versions of the described secreted proteins, as well as antisense and ribozyme molecules, can also be used in “gene therapy” approaches for the modulation of expression of the described secreted proteins. Thus, the invention also encompasses pharmaceutical formulations and methods for treating biological disorders.

Various aspects of the invention are described in greater detail in the subsections below.

### 7.1 Nucleic Acid Sequences

The cDNA sequences and corresponding deduced amino acid sequences of the described secreted proteins are presented in the Sequence Listing. The secreted protein nucleotide sequences were compiled from or obtained by: gene trapped cDNAs and clones isolated from a human testis cDNA library, and a human placenta cDNA (SEQ ID NOS:1-3); human gene trapped sequence tags (SEQ ID NOS:4-7); human gene trapped sequence tags and polynucleotides isolated from a human adrenal gland library (SEQ ID NOS:8-12); clustered human gene trapped sequences and ESTs (SEQ ID NOS:13 and 14); human gene trapped sequence tags, cDNA clones from a human mammary gland cDNA library, and the 39 N-terminal bases of human ceruloplasmin, much of which represents signal sequence that is cleaved from the precursor protein during secretion to produce a mature protein (SEQ ID NOS:15 and 16); gene trapped sequences, in conjunction with sequences available in GenBank and cDNAs isolated from human kidney mRNA (SEQ ID NOS:17-25); aligning human genomic sequences and cDNA clones from a human prostate cDNA library (SEQ ID NOS:26-30); cDNA products isolated from human testis and embryo libraries (SEQ ID NOS:31-49); aligning human genomic sequences and cDNAs made from human spleen, uterus, and trachea mRNAs (SEQ ID NOS:50-52); aligning cDNAs from pituitary and testis mRNAs and human genomic DNA sequence (SEQ ID NOS:53-57); clustered genomic sequence, ESTs, gene trapped sequence data, and cDNAs from mammary gland, thyroid, adipose, lymph node, testis, skeletal muscle, kidney, esophagus, heart, placenta, and bone marrow mRNAs (SEQ ID NOS:58-61); aligning cDNAs from thymus mRNAs and human genomic DNA sequence (SEQ ID NOS:62-66); aligning cDNAs from gene trapped human cells, and adipose and testis mRNAs, and human genomic DNA sequence (SEQ ID NOS:67-71); genomic sequence and cDNA clones from human lymph node, adipose, placenta, cerebellum, and pituitary cDNAs (SEQ ID NOS:72-78); aligning cDNAs from brain and kidney mRNAs and human genomic DNA sequence (SEQ ID NOS:79-83); aligning cDNAs from bone marrow and skeletal muscle mRNAs and human genomic P DNA sequence (SEQ ID NOS:84-86); aligning cDNAs made from testis and human fetal mRNA and human genomic DNA sequence (SEQ ID NOS:87-89); clustered genomic sequence, ESTs, and cDNAs produced using human brain, lymph node, fetal kidney, fetal lung, and hypothalamus mRNAs (SEQ ID NOS:90-101); clustered genomic sequence, ESTs, and cDNAs generated from human lymph node, liver, spleen, and fetal kidney mRNAs (SEQ ID NOS:102-103); aligning cDNAs from human kidney, fetal kidney, prostate, and lymph node mRNAs and human genomic DNA sequence (SEQ ID NOS:104-128); human genomic sequence and cDNAs made from human fetal lung and lymph node mRNAs (SEQ ID NOS:129-132); and aligning cDNAs from human brain, skeletal muscle, liver, testis, placenta, lung, bone marrow, lymph node, and prostate mRNAs and human genomic DNA sequence (SEQ ID NOS:133-136). mRNA and cDNA libraries were purchased from Clontech (Palo Alto, Calif.) and/or Edge Biosystems (Gaithersburg, Md.).

The described sequences are apparently encoded on: human chromosome 17 (SEQ ID NOS:26-30); human chromosome 10 (SEQ ID NOS:50-52); human chromosome 9, see GenBank Accession Number AC008888 (SEQ ID NOS:53-57); human chromosome 1, see GenBank Accession Number AF156100 (SEQ ID NOS:58-61); human chromosome 13, see GenBank Accession Number AL137780 (SEQ ID NOS:67-71); human chromosome 9, see GenBank Accession Number AL354982 (SEQ ID NOS:72-78); human chromosome 17, see GenBank Accession Number AC019316 (SEQ ID NOS:79-83); human chromosome 1 or both of human chromosomes 4 and 6, see GenBank Accession Numbers AC048370 and AC016488 (SEQ ID NOS:84-86); human chromosome 1, see GenBank Accession Number AL356323 (SEQ ID NOS:87-89); human chromosome 1, see GenBank Accession Number AL359826 (SEQ ID NOS:90-102); multiple exons interspersed on human chromosome 11, see GenBank Accession Number AC090384 (SEQ ID NOS:102-103); human chromosome 7, see GenBank Accession Number AC024952 (SEQ ID NOS:104-128); several exons dispersed on human chromosome 1, see GenBank Accession Number AL138787 (SEQ ID NOS:129-132); and human chromosome 7, see GenBank Accession Number AC009262 (SEQ ID NOS:133-136). As such, the described sequences are useful for mapping the coding region of the human genome, and for identifying exon splice junctions (which can, among other things, have direct application in forensic studies).

A number of polymorphisms were identified during the sequencing of the described nucleotide sequences, including: a transcriptionally silent C-to-T transition at nucleotide (nt) position 81 of SEQ ID NO:1, both of which result in an asparagine residue at corresponding amino acid (aa) position 27 of SEQ ID NO:2; a G-to-C transversion at nt position 965 of SEQ ID NO:1, which can result in a serine or threonine residue at corresponding aa position 322 of SEQ ID NO:2; a C-to-G transversion at nt position 165 of the 5′ UTR of SEQ ID NO:3; an A-to-G transition at nt position 598 of SEQ ID NO:13, which can result in an isoleucine or valine residue at corresponding aa position 200 of SEQ ID NO:14; a G-to-A transition at nt position 1756 of SEQ ID NO:15 (denoted by an “r” in the Sequence Listing), which can result in a valine or isoleucine residue at corresponding aa position 586 of SEQ ID NO:16; a G-to-C transversion at nt position 212 of SEQ ID NOS:17 and 19, and nt position 236 of SEQ ID NOS:21 and 23 (denoted by an “s” in the Sequence Listing), which can result in a glycine or alanine residue at corresponding aa position 71 of SEQ ID NOS:18 and 20, and aa position 79 of SEQ ID NOS:22 and 24; an A-to-C transversion at nt position 219 of SEQ ID NOS:17 and 19, and nt position 243 of SEQ ID NOS:21 and 23 (denoted by an “m” in eh Sequence Listing), which can result in a lysine or asparagine residue at corresponding aa position 73 of SEQ ID NOS:18 and 20, and aa position 81 of SEQ ID NOS:22 and 24; a silent G-to-A transition at nt position 30 of SEQ ID NOS:21 and 23 (denoted by an “r” in the Sequence Listing), both of which result in a glutamine residue at corresponding aa position 10 of SEQ ID NOS:22 and 24; a C/G transversion at nt position 242 of SEQ ID NOS:53 and 55, which can result in an alanine or glycine residue at corresponding aa position 81 of SEQ ID NOS:54 and 56; a T/G transversion at nt position 289 of SEQ ID NOS:53 and 55, which can result in a leucine or valine residue at corresponding aa position 97 of SEQ ID NOS:54 and 56; a T/C polymorphism at nt position 397 of SEQ ID NO:58 (denoted by a “y” in the Sequence Listing), which can result in a serine or proline residue at corresponding aa position 133 of SEQ ID NO:59; a T/A polymorphism at nt position 1124 of SEQ ID NO:58 (denoted by a “w” in the Sequence Listing), which can result in an isoleucine or asparagine residue at corresponding aa position 375 of SEQ ID NO:59; an A/G polymorphism at nt position 2072 of SEQ ID NO:58 (denoted by an “r” in the Sequence Listing), which can result in a lysine or arginine residue at corresponding aa position 691 of SEQ ID NO:59; a C/T polymorphism at nt position 2513 of SEQ ID NO:58 (denoted by a “y” in the Sequence Listing), which can result in a proline or leucine residue at corresponding aa position 838 of SEQ ID NO:59; a T/C polymorphism at nt position 3244 of SEQ ID NO:58 (denoted by a “y” in the Sequence Listing), which can result in a serine or proline residue at corresponding aa position 1082 of SEQ ID NO:59; an A/G polymorphism at nt position 3787 of SEQ ID NO:58 (denoted by an “r” in the Sequence Listing), which can result in a threonine or alanine residue at corresponding aa position 1263 of SEQ ID NO:59; a silent A/G polymorphism at nt position 4665 of SEQ ID NO:58, and nt position 489 of SEQ ID NO:60 (denoted by an “r” in the Sequence Listing), both of which result in a threonine residue at corresponding aa position 1555 of SEQ ID NO:59, and aa position 163 of SEQ ID NO:61; an A/C polymorphism at nt position 4667 of SEQ ID NO:58, and nt position 491 of SEQ ID NO:60 (denoted by an “m” in the Sequence Listing), which can result in an aspartate or alanine residue at corresponding aa position 1556 of SEQ ID NO:59, and aa position 164 of SEQ ID NO:61; a silent T/C polymorphism at nt position 4857 of SEQ ID NO:58, and nt position 681 of SEQ ID NO:60 (denoted by a “y” in the Sequence Listing), both of which result in a histidine residue at corresponding aa position 1619 of SEQ ID NO:59, and aa position 227 of SEQ ID NO:61; a T/C polymorphism at nt position 6734 of SEQ ID NO:58, and nt position 2558 of SEQ ID NO:60 (denoted by a “y” in the Sequence Listing), which can result in a valine or alanine residue at corresponding aa position 2245 of SEQ ID NO:59, and aa position 853 of SEQ ID NO:61; a T/C polymorphism at nt position 7253 of SEQ ID NO:58, and nt position 3077 of SEQ ID NO:60 (denoted by a “y” in the Sequence Listing), which can result in an isoleucine or threonine residue at corresponding aa position 2418 of SEQ ID NO:59, and aa position 1026 of SEQ ID NO:61; a silent G/C polymorphism at nt position 11940 of SEQ ID NO:58, and nt position 7764 of SEQ ID NO:60 (denoted by an “s” in the Sequence Listing), both of which result in a valine residue at corresponding aa position 3980 of SEQ ID NO:59, and aa position 2588 of SEQ ID NO:61; a T/A polymorphism at nt position 12136 of SEQ ID NO:58, and nt position 7960 of SEQ ID NO:60 (denoted by a “w” in the Sequence Listing), which can result in a serine or threonine residue at corresponding aa position 4046 of SEQ ID NO:59, and aa position 2654 of SEQ ID NO:61; a G/A polymorphism at nt position 1102 of SEQ ID NOS:72, 74, and 76, which can result in an alanine or threonine residue at corresponding aa position 368 of SEQ ID NOS:73, 75, and 77; a silent A/C polymorphism at nt position 1306 of SEQ ID NOS:72, 74, and 76, both of which result in an arginine residue at corresponding aa position 436 of SEQ ID NOS:73, 75, and 77; a C/T polymorphism at nt position 1823 of SEQ ID NOS:72, 74, and 76, which can result in an alanine or valine residue at corresponding aa position 608 of SEQ ID NOS:73, 75, and 77; an A/C polymorphism at nt position 2143 of SEQ ID NOS:72, 74, and 76, which can result in a threonine or proline residue at corresponding aa position 715 of SEQ ID NOS:73, 75, and 77; a silent A/C polymorphism at nt position 2202 of SEQ ID NOS:72, 74, and 76, both of which result in a valine residue at corresponding aa position 734 of SEQ ID NOS:73, 75, and 77; a silent A/G polymorphism at nt position 2283 of SEQ ID NOS:72, 74, and 76, both of which result in a glutamate residue at corresponding aa position 761 of SEQ ID NOS:73, 75, and 77; a G/A polymorphism at nt position 2285 of SEQ ID NOS:72, 74, and 76, which can result in a glycine or glutamate residue at corresponding aa position 762 of SEQ ID NOS:73, 75, and 77; a silent A/C polymorphism at nt position 2601 of SEQ ID NOS:72, 74, and 76, both of which result in a glycine residue at corresponding aa position 867 of SEQ ID NOS:73, 75, and 77; an A/G polymorphism at nt position 2696 of SEQ ID NOS:72, 74, and 76, which can result in a lysine or arginine residue at corresponding aa position 899 of SEQ ID NOS:73, 75, and 77; an AG/TT polymorphism at nt positions 2776-2777 of SEQ ID NOS:72, 74, and 76, which can result in a leucine or arginine residue at corresponding aa position 926 of SEQ ID NOS:73, 75, and 77; an A/C polymorphism at nt position 2873 of SEQ ID NOS:72, 74, and 76, which can result in an asparagine or threonine residue at corresponding aa position 958 of SEQ ID NOS:73, 75, and 77; a silent G/A polymorphism at nt position 3114 of SEQ ID NOS:72, 74, and 76, both of which result in a glycine residue at corresponding aa position 1038 of SEQ ID NOS:73, 75, and 77; an AT/TC polymorphism at nt positions 3115-3116 of SEQ ID NOS:72, 74, and 76, which can result in a methionine or serine residue at corresponding aa position 1039 of SEQ ID NOS:73, 75, and 77; a C/A polymorphism at nt position 4246 of SEQ ID NOS:74 and 76, which can result in a glutamine or lysine residue at corresponding aa position 1416 of SEQ ID NOS:75 and 77; a G/A polymorphism at nt position 4813 of SEQ ID NOS:74 and 76, which can result in a valine or methionine residue at corresponding aa position 1605 of SEQ ID NOS:75 and 77; a C/A polymorphism at nt position 5429 of SEQ ID NOS:74 and 76, which can result in an alanine or glutamate residue at corresponding aa position 1810 of SEQ ID NOS:75 and 77; an A/T polymorphism at nt position 5527 of SEQ ID NOS:74 and 76, which can result in a lysine residue or a STOP codon at corresponding aa position 1843 of SEQ ID NOS:75 and 77; a C/T polymorphism at nt position 6089 of SEQ ID NO:74, which can result in an alanine or valine residue at corresponding aa position 2030 of SEQ ID NO:75; a C/G polymorphism at nt position 6092 of SEQ ID NO:74, which can result in a serine or cysteine residue at corresponding aa position 2031 of SEQ ID NO:75; a C/G polymorphism at nt position 6094 of SEQ ID NO:74, which can result in a proline or alanine residue at corresponding aa position 2032 of SEQ ID NO:75; an AC/CT polymorphism at nt positions 7868-7869 of SEQ ID NO:74, which can result in an aspartate or alanine residue at corresponding aa position 2623 of SEQ ID NO:75; a silent A/G polymorphism at nt position 8250 of SEQ ID NO:74, both of which result in an alanine residue at corresponding aa position 2750 of SEQ ID NO:75; a silent T/C polymorphism at nt position 8754 of SEQ ID NO:74, both of which result in a histidine residue at corresponding aa position 2918 of SEQ ID NO:75; a C/A polymorphism at nt position 9170 of SEQ ID NO:74, which can result in a proline or histidine residue at corresponding aa position 3057 of SEQ ID NO:75; a G/T polymorphism at nt position 9176 of SEQ ID NO:74, which can result in a cysteine or phenylalanine residue at corresponding aa position 3059 of SEQ ID NO:75; a T/A polymorphism at nt position 9481 of SEQ ID NO:74, which can result in a phenylalanine or isoleucine residue at corresponding aa position 3161 of SEQ ID NO:75; a silent T/A polymorphism at nt position 9576 of SEQ ID NO:74, both of which result in a valine residue at corresponding aa position 3192 of SEQ ID NO:75; a G/A polymorphism at nt position 9625 of SEQ ID NO:74, which can result in a glutamate or lysine residue at corresponding aa position 3209 of SEQ ID NO:75; a G/A polymorphism at nt position 416 of SEQ ID NO:79, and nt position 206 of SEQ ID NO:81, which can result in an arginine or glutamine residue at corresponding aa position 139 of SEQ ID NO:80, and aa position 69 of SEQ ID NO:82; a silent C/T polymorphism at nt position 993 of SEQ ID NO:79, and nt position 783 of SEQ ID NO:81, both of which result in an alanine residue at corresponding aa position 331 of SEQ ID NO:80, and aa position 261 of SEQ ID NO:82; a C/T polymorphism at nt position 1283 of SEQ ID NO:79, and nt position 1073 of SEQ ID NO:81, which can result in a valine or alanine residue at corresponding aa position 428 of SEQ ID NO:80, and aa position 358 of SEQ ID NO:82; a silent C/T polymorphism at nt position 153 of SEQ ID NO:87, both of which result in an alanine residue at corresponding aa position 51 of SEQ ID NO:88; a C/G polymorphism at nt position 946 of SEQ ID NO:87, which can result in a glutamine or glutamate residue at corresponding aa position 316 of SEQ ID NO:88; a C/A polymorphism at nt position 953 of SEQ ID NO:87, which can result in a threonine or asparagine residue at corresponding aa position 318 of SEQ ID NO:88; a silent T/C polymorphism at nt position 513 of SEQ ID NOS:90, 94, and 98, and nt position 918 of SEQ ID NOS:92, 96, and 100 (denoted by a “y” in the Sequence Listing), both of which result in a glycine residue at corresponding aa position 171 of SEQ ID NOS:91, 95, and 99, and aa position 306 of SEQ ID NOS:93, 97, and 101; a T/C polymorphism at nt position 938 of SEQ ID NOS:90, 94, and 98, and nt position 1343 of SEQ ID NOS:92, 96, and 100 (denoted by a “y” in the Sequence Listing), which can result in a valine or alanine residue at corresponding aa position 313 of SEQ ID NOS:91, 95, and 99, and aa position 448 of SEQ ID NOS:93, 97, and 101; a silent A/C polymorphism at nt position 1068 of SEQ ID NOS:90, 94, and 98, and nt position 1473 of SEQ ID NOS:92, 96, and 100 (denoted by an “m” in the Sequence Listing), both of which result in a threonine residue at corresponding aa position 356 of SEQ ID NOS:91, 95, and 99, and aa position 491 of SEQ ID NOS:93, 97, and 101; a C/G polymorphism at nt position 2562 of SEQ ID NO:90, and nt position 2967 of SEQ ID NO:92 (denoted by an “s” in the Sequence Listing), which can result in an aspartate or glutamate residue at corresponding aa position 854 of SEQ ID NO:91, and aa position 989 of SEQ ID NO:93; a silent T/C polymorphism at nt position 2640 of SEQ ID NO:90, and nt position 3045 of SEQ ID NO:92 (denoted by a “y” in the Sequence Listing), both of which result in a phenylalanine residue at corresponding aa position 880 of SEQ ID NO:91, and aa position 1015 of SEQ ID NO:93; a G/T polymorphism at nt position 92 of SEQ ID NOS:92, 96, and 100 (denoted by a “k” in the Sequence Listing), which can result in an arginine or leucine residue at corresponding aa position 31 of SEQ ID NOS:93, 97, and 101; a silent T/C polymorphism at nt position 120 of SEQ ID NOS:92, 96, and 100 (denoted by a “y” in the Sequence Listing), both of which result in a proline residue at corresponding aa position 40 of SEQ ID NOS:93, 97, and 101; a C/G polymorphism at nt position 1852 of SEQ ID NO:94, and nt position 2257 of SEQ ID NO:96 (denoted by an “s” in the Sequence Listing), which can result in an alanine or proline residue at corresponding aa position 618 of SEQ ID NO:95, and aa position 753 of SEQ ID NO:97; a silent A/C polymorphism at nt position 2085 of SEQ ID NO:94, and nt position 2490 of SEQ ID NO:96 (denoted by an “m” in the Sequence Listing), both of which result in an alanine at corresponding aa position 695 of SEQ ID NO:95, and aa position 830 of SEQ ID NO:97; a T/C polymorphism at nt position 1822 of SEQ ID NO:98, and nt position 2227 of SEQ ID NO:100 (denoted by a “y” in the Sequence Listing), which can result in a cysteine or arginine residue at corresponding aa position 608 of SEQ ID NO:99, and aa position 743 of SEQ ID NO:101; a silent A/C polymorphism at nt position 1866 of SEQ ID NO:98, and nt position 2271 of SEQ ID NO:100 (denoted by an “m” in the Sequence Listing), both of which result in a leucine residue at corresponding aa position 622 of SEQ ID NO:99, and aa position 757 of SEQ ID NO:101; a T/C polymorphism at nt position 2063 of SEQ ID NO:98, and nt position 2468 of SEQ ID NO:100 (denoted by a “y” in the Sequence Listing), which can result in a leucine or proline at corresponding aa position 688 of SEQ ID NO:99, and aa position 823 of SEQ ID NO:101; a G/C polymorphism at nt position 81 of SEQ ID NO:102, which can result in an arginine or serine residue at corresponding aa position 27 of SEQ ID NO:103; a T/A polymorphism at nt position 550 of SEQ ID NOS:104 and 106, and nt position 349 of SEQ ID NOS:114 and 116, which can result in a cysteine or serine residue at corresponding aa position 184 of SEQ ID NOS:105 and 107, and aa position 117 of SEQ ID NOS:115 and 1.17; a G/A polymorphism at nt position 274 of SEQ ID NO:129, and nt position 232 of SEQ ID NO:131, which can result in a glutamate or lysine residue at corresponding aa position 92 of SEQ ID NO:130, and aa position 78 of SEQ ID NO:132; a C/A polymorphism at nt position 424 of SEQ ID NO:129, and nt position 382 of SEQ ID NO:131, which can result in a proline or threonine residue at corresponding aa position 142 of SEQ ID NO:130, and aa position 128 of SEQ ID NO:132; a silent C/T polymorphism at nt position 732 of SEQ ID NO:129, and nt position 690 of SEQ ID NO:131, both of which result in leucine residue at corresponding aa position 244 of SEQ ID NO:130, and aa position 230 of SEQ ID NO:132; a G/A polymorphism at nt position 787 of SEQ ID NO:129, and nt position 745 of SEQ ID NO:131, which can result in a glycine or arginine residue at corresponding aa position 263 of SEQ ID NO:130, and aa position 249 of SEQ ID NO:132; a G/A polymorphism at nt position 1090 of SEQ ID NO:129, and nt position 1048 of SEQ ID NO:131, which can result in a glutamate or lysine residue at corresponding aa position 364 of SEQ ID NO:130, and aa position 350 of SEQ ID NO:132; a silent T/C polymorphism at nt position 408 of SEQ ID NOS:133 and 135, both of which result in a glycine residue at corresponding aa position 136 of SEQ ID NOS:134 and 136; an A/C polymorphism at nt position 553 of SEQ ID NOS:133 and 135, which can result in a lysine or glutamine residue at corresponding aa position 185 of SEQ ID NOS:134 and 136; a silent T/G polymorphism at nt position 1461 of SEQ ID NO:133, and nt position 1287 of SEQ ID NO:135, both of which result in a proline residue at corresponding aa position 487 of SEQ ID NO:134, and aa position 429 of SEQ ID NO:136; a silent C/G polymorphism at nt position 1935 of SEQ ID NO:133, and nt position 1761 of SEQ ID NO:135, both of which result in a threonine residue at corresponding aa position 645 of SEQ ID NO:134, and aa position 587 of SEQ ID NO:136; and a silent C/T polymorphism at nt position 2028 of SEQ ID NO:133, and nt position 1854 of SEQ ID NO:135, both of which result in a cysteine residue at corresponding aa position 676 of SEQ ID NO:134, and aa position 618 of SEQ ID NO:136. The present invention contemplates sequences comprising any and all combinations and permutations of the above polymorphisms. As these polymorphisms are coding single nucleotide polymorphisms (SNPs), they are particularly useful in forensic analysis.

An additional application of the described novel human polynucleotide sequences is their use in the molecular mutagenesis/evolution of proteins that are at least partially encoded by the described novel sequences using, for example, polynucleotide shuffling or related methodologies. Such approaches are described in U.S. Pat. Nos. 5,830,721 and 5,837,458.

The described secreted protein gene products can also be expressed in transgenic animals. Animals of any non-human species, including, but not limited to, worms, mice, rats, rabbits, guinea pigs, pigs, micro-pigs, birds, goats, and non-human primates, e.g., baboons, monkeys, and chimpanzees, may be used to generate transgenic animals comprising the described secreted protein sequences.

Any technique known in the art may be used to introduce a secreted protein transgene into animals to produce the founder lines of transgenic animals. Such techniques include, but are not limited to: pronuclear microinjection (U.S. Pat. No. 4,873,191); retrovirus-mediated gene transfer into germ lines (Van der Putten et al., Proc. Natl. Acad. Sci. USA 82:6148-6152, 1985); gene targeting in embryonic stem cells (Thompson et al., Cell 56:313-321, 1989); electroporation of embryos (Lo, Mol. Cell. Biol. 3:1803-1814, 1983); and sperm-mediated gene transfer (Lavitrano et al., Cell 57:717-723, 1989); etc. For a review of such techniques, see, e.g., Gordon, Intl. Rev. Cytol. 115:171-229, 1989.

The present invention provides for transgenic animals that carry a secreted protein transgene in all their cells, as well as animals that carry a transgene in some, but not all their cells, i.e., mosaic animals or somatic cell transgenic animals. A transgene may be integrated as a single transgene, or in concatamers, e.g., head-to-head tandems or head-to-tail tandems. A transgene may also be selectively introduced into and activated in a particular cell-type by following, for example, the teaching of Lakso et al., Proc. Natl. Acad. Sci. USA 89:6232-6236, 1992. The regulatory sequences required for such a cell-type specific activation will depend upon the particular cell-type of interest, and will be apparent to those of skill in the art.

When it is desired that a secreted protein transgene be integrated into the chromosomal site of the endogenous gene encoding the secreted protein, gene targeting is preferred. Briefly, when such a technique is to be utilized, vectors containing some nucleotide sequences homologous to the endogenous gene encoding the secreted protein are designed for the purpose of integrating, via homologous recombination with chromosomal sequences, into and disrupting the function of the nucleotide sequence of the endogenous gene encoding the secreted protein (i.e., “knockout” animals).

The transgene can also be selectively introduced into a particular cell-type, thus inactivating the endogenous gene encoding the secreted protein in only that cell-type, by following, for example, the teaching of Gu et al., Science 265:103-106, 1994. The regulatory sequences required for such a cell-type specific inactivation will depend upon the particular cell-type of interest, and will be apparent to those of skill in the art.

Once transgenic animals have been generated, the expression of the recombinant gene encoding the secreted protein may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques that include, but are not limited to, Northern blot analysis of tissue samples obtained from the animal, in situ hybridization analysis, and RT-PCR. Samples of secreted protein gene-expressing tissue may also be evaluated immunocytochemically using antibodies specific for the secreted protein transgene product.

The present invention also provides for “knock-in” animals. Knock-in animals are those in which a polynucleotide sequence (i.e., a gene or a cDNA) that the animal does not naturally have in its genome is inserted in such a way that it is expressed. Examples include, but are not limited to, a human gene or cDNA used to replace its murine ortholog in the mouse, a murine cDNA used to replace the murine gene in the mouse, and a human gene or cDNA or murine cDNA that is tagged with a reporter construct used to replace the murine ortholog or gene in the mouse. Such replacements can occur at the locus of the murine ortholog or gene, or at another specific site. Such knock-in animals are useful for the in vivo study, testing and validation of, intra alia, human drug targets, as well as for compounds that are directed at the same, and therapeutic proteins.

### 7.2 Amino Acid Sequences

The described secreted proteins, polypeptides, peptide fragments, mutated, truncated, or deleted forms of the described secreted proteins, and/or secreted protein fusion proteins can be prepared for a variety of uses. These uses include, but are not limited to, the generation of antibodies, as reagents in diagnostic assays, for the identification of other cellular gene products related to the described secreted proteins, and as reagents in assays for screening for compounds that can be used as pharmaceutical reagents useful in the therapeutic treatment of mental, biological, or medical disorders and diseases. Given the similarity information and expression data, the described secreted proteins can be targeted (by drugs, oligonucleotides, antibodies, etc.) in order to treat disease, or to augment the efficacy of, for example, chemotherapeutic agents used in the treatment of cancer, such as breast or prostate cancer, and therapeutic agents used in the treatment of, for example, inflammatory disorders, arthritis, or infectious diseases, as antiviral agents, or to promote healing.

The Sequence Listing discloses the amino acid sequences encoded by the described secreted protein sequences. The described secreted protein sequences display initiator methionines in DNA sequence contexts consistent with translation initiation sites, and nearly all incorporate hydrophobic sequences similar to those found in membrane and secreted proteins.

As putative secreted proteins/peptides, signal peptides associated with the described amino acid sequences may be typically cleaved during secretion of the mature protein products. Analysis of the described proteins/peptides reveals the presence of predicted signal cleavage sites between about 13 and about 53 amino acids into the described proteins (from the initiation methionine). For example, SEQ ID NO:85 displays a predicted cleavage site at or around amino acid positions 25 or 26, which indicates the approximate position of the N-terminus of the processed, or “mature,” form of the protein after cleavage by eucaryotic secretion machinery. Computer predictions of signal peptidase cleavage sites being less than absolutely accurate, an additional aspect of the present invention includes any and all mature cleavage products remaining after removal of between about the first 10 and about the first 55 amino acids, or any number in-between (as applicable given the length of the described protein), that leaves (for secretion) at least about 3, and preferably at least about 6 to 20, or more, amino acids of the protein product originally encoded by the described sequences (for secretion).

The secreted protein amino acid sequences of the invention include the amino acid sequences presented in the Sequence Listing, as well as analogues and derivatives thereof. Further, corresponding secreted protein homologues from other species are encompassed by the invention. In fact, any product encoded by the secreted protein nucleotide sequences described herein are within the scope of the invention, as are any novel polynucleotide sequences encoding all or any novel portion of an amino acid sequence presented in the Sequence Listing. The degenerate nature of the genetic code is well-known, and, accordingly, each amino acid presented in the Sequence Listing is generically representative of the well-known nucleic acid “triplet” codon, or in many cases codons, that can encode the amino acid. As such, as contemplated herein, the amino acid sequences presented in the Sequence Listing, when taken together with the genetic code (see, for example, “Molecular Cell Biology”, Table 4-1 at page 109 (Darnell et al., eds., Scientific American Books, New York, N.Y., 1986)), are generically representative of all the various permutations and combinations of nucleic acid sequences that can encode such amino acid sequences.

The invention also encompasses proteins that are functionally equivalent to the secreted proteins encoded by the presently described nucleotide sequences, as judged by any of a number of criteria, including, but not limited to, the ability to bind and cleave a substrate of the described secreted proteins, the ability to effect an identical or complementary downstream pathway, or a change in cellular metabolism (e.g., proteolytic activity, ion flux, tyrosine phosphorylation, etc.). Such functionally equivalent secreted proteins include, but are not limited to, additions or substitutions of amino acid residues within the amino acid sequence encoded by the secreted protein nucleotide sequences described herein, but that result in a silent change, thus producing a functionally equivalent expression product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

A variety of host-expression vector systems can be used to express the secreted protein nucleotide sequences of the invention. Where, as in the present instance, the peptides or polypeptides are thought to be soluble or secreted molecules, a peptide or polypeptide can be recovered from the culture media. Such expression systems also encompass engineered host cells that express the described secreted proteins, or functional equivalents, in situ. Purification or enrichment of the described secreted proteins from such expression systems can be accomplished using appropriate detergents and lipid micelles and methods well-known to those skilled in the art. However, such engineered host cells themselves may be used in situations where it is important not only to retain the structural and functional characteristics of the described secreted proteins, but to assess biological activity, e.g., in certain drug screening assays.

The expression systems that may be used for purposes of the invention include, but are not limited to, microorganisms such as bacteria (e.g., E. coli, B. subtilis) transformed with recombinant bacteriophage DNA, plasmid DNA or cosmid DNA expression vectors containing the described secreted protein nucleotide sequences; yeast (e.g., Saccharomyces, Pichia) transformed with recombinant yeast expression vectors containing the described secreted protein nucleotide sequences; insect cell systems infected with recombinant virus expression vectors (e.g., baculovirus) containing the described secreted protein nucleotide sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the described secreted protein nucleotide sequences; or mammalian cell systems (e.g., COS, CHO, BHK, 293, 3T3) harboring recombinant expression constructs containing the described secreted protein nucleotide sequences and promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the secreted protein product being expressed. For example, when a large quantity of such a protein is to be produced for the generation of pharmaceutical compositions of or containing the described secreted proteins, or for raising antibodies to the described secreted proteins, vectors that direct the expression of high levels of fusion protein products that are readily purified may be desirable. Such vectors include, but are not limited to, the E. coli expression vector pUR278 (Ruther and Muller-Hill, EMBO J. 2:1791-1794, 1983), in which the described secreted protein coding sequences may be ligated individually into the vector in-frame with the lacZ coding region so that a fusion protein is produced; pIN vectors (Inouye and Inouye, Nucl. Acids Res. 13:3101-3109, 1985; Van Heeke and Schuster, J. Biol. Chem. 264:5503-5509, 1989); and the like. PGEX vectors (Pharmacia or American Type Culture Collection) can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target expression product can be released from the GST moiety.

In an exemplary insect system, Autographa californica nuclear polyhedrosis virus (AcNPV) is used as a vector to express foreign polynucleotide sequences. The virus grows in Spodoptera frugiperda cells. A secreted protein coding sequence can be cloned individually into a non-essential region (for example the polyhedrin gene) of the virus and placed under control of an AcNPV promoter (for example the polyhedrin promoter). Successful insertion of a secreted protein coding sequence will result in inactivation of the polyhedrin gene and production of non-occluded recombinant virus (i.e., virus lacking the proteinaceous coat coded for by the polyhedrin gene), These recombinant viruses are then used to infect Spodoptera frugiperda cells in which the inserted sequence is expressed (see, e.g., Smith et al., J. Virol. 46:584-593, 1983, and U.S. Pat. No. 4,215,051).

In mammalian host cells, a number of viral-based expression systems can be utilized. In cases where an adenovirus is used as an expression vector, the secreted protein nucleotide sequence of interest may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing a secreted protein product in infected hosts (see, e.g., Logan and Shenk, Proc. Natl. Acad. Sci. USA 81:3655-3659, 1984). Specific initiation signals may also be required for efficient translation of inserted secreted protein nucleotide sequences. These signals include the ATG initiation codon and adjacent sequences. In cases where an entire secreted protein gene or cDNA, including its own initiation codon and adjacent sequences, is inserted into the appropriate expression vector, no additional translational control signals may be needed. However, in cases where only a portion of a secreted protein coding sequence is inserted, exogenous translational control signals, including, perhaps, the ATG initiation codon, may be provided. Furthermore, the initiation codon should be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bitter et al., Methods in Enzymol. 153:516-544, 1987).

In addition, a host cell strain may be chosen that modulates the expression of the inserted sequences, or modifies and processes the expression product in the specific fashion desired. Such modifications (e.g., glycosylation) and processing (e.g., cleavage) of protein products may be important for the function of the protein. Different host cells have characteristic and specific mechanisms for the post-translational processing and modification of proteins and expression products. Appropriate cell lines or host systems can be chosen to ensure the desired modification and processing of the foreign protein expressed. To this end, eukaryotic host cells that possess the cellular machinery for the desired processing of the primary transcript, glycosylation, and phosphorylation of the expression product may be used. Such mammalian host cells include, but are not limited to, CHO, VERO, BHK, HeLa, COS, MDCK, 293, 3T3, WI38, and in particular, human cell lines.

For long-term, high-yield production of recombinant proteins, stable expression is preferred. For example, cell lines that stably express the secreted protein sequences described herein can be engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express the described secreted protein products. Such engineered cell lines may be particularly useful in screening and evaluation of compounds that affect the endogenous activity of the described secreted protein products.

A number of selection systems may be used, including, but not limited to, the herpes simplex virus thymidine kinase (Wigler et al., Cell 11:223-232, 1977), hypoxanthine-guanine phosphoribosyltransferase (Szybalska and Szybalski, Proc. Natl. Acad. Sci. USA 48:2026-2034, 1962), and adenine phosphoribosyltransferase (Lowy et al., Cell 22:817-823, 1980) genes, which can be employed in tk⁻, hgprt⁻ or aprt⁻ cells, respectively. Also, antimetabolite resistance can be used as the basis of selection for the following genes: dihydrofolate reductase (dhfr), which confers resistance to methotrexate (Wigler et al., Proc. Natl. Acad. Sci. USA 77:3567-3570, 1980, and O'Hare et al., Proc. Natl. Acad. Sci. USA 78:1527-1531, 1981); guanine phosphoribosyl transferase (gpt), which confers resistance to mycophenolic acid (Mulligan and Berg, Proc. Natl. Acad. Sci. USA 78:2072-2076, 1981); neomycin phosphotransferase (neo), which confers resistance to G-418 (Colbere-Garapin et al., J. Mol. Biol. 150:1-14, 1981); and hygromycin B phosphotransferase (hpt), which confers resistance to hygromycin (Santerre et al., Gene 30:147-156, 1984).

Alternatively, any fusion protein can be readily purified by utilizing an antibody specific for the fusion protein being expressed. Another exemplary system allows for the ready purification of non-denatured fusion proteins expressed in human cell lines (Janknecht et al., Proc. Natl. Acad. Sci. USA 88:8972-8976, 1991). In this system, the sequence of interest is subcloned into a vaccinia recombination plasmid such that the sequence's open reading frame is translationally fused to an amino-terminal tag consisting of six histidine residues. Extracts from cells infected with recombinant vaccinia virus are loaded onto Ni²⁺-nitriloacetic acid-agarose columns, and histidine-tagged proteins are selectively eluted with imidazole-containing buffers.

Also encompassed by the present invention are fusion proteins that direct the described secreted proteins to a target organ and/or facilitate transport across the membrane into the cytosol. Conjugation of the described secreted proteins to antibody molecules or their Fab fragments could be used to target cells bearing a particular epitope. Attaching an appropriate signal sequence to the described secreted proteins would also transport the described secreted proteins to a desired location within the cell. Alternatively, targeting of the described secreted proteins or nucleic acid sequences might be achieved using liposome or lipid complex based delivery systems. Such technologies are described in “Liposomes: A Practical Approach” (New, R.R. C., ed., IRL Press, New York; NY, 1990), and in U.S. Pat. Nos. 4,594,595, 5,459,127, 5,948,767 and 6,110,490. Additionally embodied are novel protein constructs engineered in such a way that they facilitate transport of the described secreted proteins to a target site or desired organ, where they cross the cell membrane and/or the nucleus, where the described secreted proteins can exert their functional activity. This goal may be achieved by coupling of the described secreted proteins to a cytokine or other ligand that provides targeting specificity, and/or to a protein transducing domain (see generally U.S. Provisional Patent Application Ser. Nos. 60/111,701 and 60/056,713, for examples of such transducing sequences), to facilitate passage across cellular membranes, and can optionally be engineered to include nuclear localization signals.

Additionally contemplated are oligopeptides that are modeled on an amino acid sequence first described in the Sequence Listing. Such secreted protein oligopeptides are generally between about 10 to about 100 amino acids long, or between about 16 to about 80 amino acids long, or between about 20 to about 35 amino acids long, or any variation or combination of sizes represented therein that incorporate a contiguous region of sequence first disclosed in the Sequence Listing. Such secreted protein oligopeptides can be of any length disclosed within the above ranges and can initiate at any amino acid position represented in the Sequence Listing.

The invention also contemplates “substantially isolated” or “substantially pure” proteins or polypeptides. By a “substantially isolated” or “substantially pure” protein or polypeptide is meant a protein or polypeptide that has been separated from at least some of those components that naturally accompany it. Typically, the protein or polypeptide is substantially isolated or pure when it is at least 60%, by weight, free from the proteins and other naturally-occurring organic molecules with which it is naturally associated in vivo. Preferably, the purity of the preparation is at least 75%, more preferably at least 90%, and most preferably at least 99%, by weight. A substantially isolated or pure protein or polypeptide may be obtained, for example, by extraction from a natural source, by expression of a recombinant nucleic acid encoding the protein or polypeptide, or by chemically synthesizing the protein or polypeptide.

Purity can be measured by any appropriate method, e.g., column chromatography such as immunoaffinity chromatography using an antibody specific for the protein or polypeptide, polyacrylamide gel electrophoresis, or HPLC analysis. A protein or polypeptide is substantially free of naturally associated components when it is separated from at least some of those contaminants that accompany it in its natural state. Thus, a polypeptide that is chemically synthesized or produced in a cellular system different from the cell from which it naturally originates will be, by definition, substantially free from its naturally associated components. Accordingly, substantially isolated or pure proteins or polypeptides include eukaryotic proteins synthesized in E. coli, other prokaryotes, or any other organism in which they do not naturally occur.

### 7.3 Antibodies to the Described Secreted Proteins

Antibodies that specifically recognize one or more epitopes of the described secreted proteins, epitopes of conserved variants of the described secreted proteins, or peptide fragments of the described secreted proteins, are also encompassed by the invention. Such antibodies include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the invention may be used, for example, in the detection of the described secreted proteins in a biological sample and may, therefore, be utilized as part of a diagnostic or prognostic technique whereby patients may be tested for abnormal amounts of the described secreted proteins. Such antibodies may also be utilized in conjunction with, for example, compound screening schemes for the evaluation of the effect of test compounds on expression and/or activity of the described secreted proteins. Additionally, such antibodies can be used in conjunction with gene therapy to, for example, evaluate normal and/or engineered secreted protein-expressing cells prior to their introduction into a patient. Such antibodies may additionally be used in methods for the inhibition of abnormal activity of the described secreted proteins. Thus, such antibodies may be utilized as a part of treatment methods.

For the production of antibodies, various host animals may be immunized by injection with the described secreted proteins, peptides (e.g., corresponding to a functional domain of the described secreted proteins), truncated polypeptides (the described secreted proteins in which one or more domains have been deleted), functional equivalents of the described secreted proteins or mutated variants of the described secreted proteins. Such host animals may include, but are not limited to, pigs, rabbits, mice, goats, and rats, to name but a few. Various adjuvants may be used to increase the immunological response, depending on the host species, including, but not limited to, Freund's adjuvant (complete and incomplete), mineral salts such as aluminum hydroxide or aluminum phosphate, chitosan, surface active substances such as lysolecithin, pluronic polyols, polyanions, peptides, oil emulsions, and potentially useful human adjuvants such as BCG (bacille Calmette-Guerin) and Corynebacterium parvum. Alternatively, the immune response could be enhanced by combination and/or coupling with molecules such as keyhole limpet hemocyanin, tetanus toxoid, diphtheria toxoid, ovalbumin, cholera toxin, or fragments thereof. Polyclonal antibodies are heterogeneous populations of antibody molecules derived from the sera of the immunized animals.

Monoclonal antibodies, which are homogeneous populations of antibodies to a particular antigen, can be obtained by any technique that provides for the production of antibody molecules by continuous cell lines in culture. These include, but are not limited to, the hybridoma technique (Kohler and Milstein, Nature 256:495-497, 1975, and U.S. Pat. No. 4,376,110), the human B-cell hybridoma technique (Kosbor et al., Immunology Today 4:72, 1983, and Cote et al., Proc. Natl. Acad. Sci. USA 80:2026-2030, 1983), and the EBV-hybridoma technique (Cote et al., in “Monoclonal Antibodies and Cancer Therapy”, Vol. 27, UCLA Symposia on Molecular and Cellular Biology, New Series, pp. 77-96 (Reisfeld and Sell, eds., Alan R. Liss, Inc. New York, N.Y., 1985)). Such antibodies may be of any immunoglobulin class, including IgG, IgM, IgE, IgA, and IgD, and any subclass thereof. The hybridomas producing the mAbs of this invention may be cultivated in vitro or in vivo. Production of high titers of mabs in vivo makes this the presently preferred method of production.

In addition, techniques developed for the production of “chimeric antibodies” (Morrison et al., Proc. Natl. Acad. Sci. USA 81:6851-6855, 1984, Neuberger et al., Nature 312:604-608, 1984, and Takeda et al., Nature 314:452-454, 1985) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. Such technologies are described in U.S. Pat. Nos. 6,114,598, 6,075,181 and 5,877,397. Also encompassed by the present invention is the use of fully humanized monoclonal antibodies, as described in U.S. Pat. No. 6,150,584.

Alternatively, techniques described for the production of single chain antibodies (U.S. Pat. No. 4,946,778, Bird, Science 242:423-426, 1988, Huston et al., Proc. Natl. Acad. Sci. USA 85:5879-5883, 1988, and Ward et al., Nature 341:544-546, 1989) can be adapted to produce single chain antibodies against the described secreted protein expression products. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include, but are not limited to: F(ab′)₂ fragments, which can be produced by pepsin digestion of an antibody molecule; and Fab fragments, which can be generated by reducing the disulfide bridges of F(ab′)₂ fragments. Alternatively, Fab expression libraries may be constructed (Huse et al., Science 246:1275-1281, 1989) to allow rapid and easy identification of monoclonal Fab fragments with the desired specificity.

Antibodies to the described secreted proteins can, in turn, be utilized to generate anti-idiotype antibodies that “mimic” the described secreted proteins, using techniques well-known to those skilled in the art (see, e.g., Greenspan and Bona, FASEB J. 7:437-444, 1993, and Nisonoff, J. Immunol. 147:2429-2438, 1991). For example, antibodies that bind to a domain of the described secreted proteins and competitively inhibit the binding of the described secreted proteins to their cognate receptors can be used to generate anti-idiotypes that “mimic” the described secreted proteins and, therefore, bind and activate or neutralize a receptor. Such anti-idiotypic antibodies, or Fab fragments of such anti-idiotypes, can be used in therapeutic regimens involving a signaling pathway involving the described secreted proteins.

Additionally given the high degree of relatedness of mammalian proteins, the described secreted proteins knock-out mice (having never seen the described secreted proteins, and thus never been tolerized to the described secreted proteins) have an unique utility, as they can be advantageously applied to the generation of antibodies against the disclosed mammalian secreted proteins (i.e., the described secreted proteins will be immunogenic in the described secreted proteins knock-out animals).

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All cited publications, patents, and patents applications, are herein incorporated by reference in their entirety.

1. An isolated nucleic acid molecule that encodes the amino acid sequence of SEQ ID NO:2, 5, 7, 9, 11, 14, 16, 18, 20, 22, 24, 27, 29, 32, 34, 36, 38, 40, 42, 44, 46, 48, 51, 54, 56, 59, 61, 63, 65, 68, 70, 73, 75, 77, 80, 82, 85, 88, 91, 93, 95, 97, 99, 101, 103, 105, 107, 109, 111, 113, 115, 117, 119, 121, 123, 125, 127, 130, 132, 134, or 136.

2. The isolated nucleic acid molecule of claim 1, wherein said nucleic acid molecule comprises the nucleotide sequence of SEQ ID NO:1, 4, 6, 8, 10, 13, 15, 17, 19, 21, 23, 26, 28, 31, 33, 35, 37, 39, 41, 43, 45, 47, 50, 53, 55, 58, 60, 62, 64, 67, 69, 72, 74, 76, 79, 81, 84, 87, 90, 92, 94, 96, 98, 100, 102, 104, 106, 108, 110, 112, 114, 116, 118, 120, 122, 124, 126, 129, 131, 133, or 135.

3. An expression vector comprising the isolated nucleic acid molecule of claim 1.

4. An isolated polypeptide comprising the amino acid sequence of SEQ ID NO:2, 5, 7, 9, 11, 14, 16, 18, 20, 22, 24, 27, 29, 32, 34, 36, 38, 40, 42, 44, 46, 48, 51, 54, 56, 59, 61, 63, 65, 68, 70, 73, 75, 77, 80, 82, 85, 88, 91, 93, 95, 97, 99, 101, 103, 105, 107, 109, 111, 113, 115, 117, 119, 121, 123, 125, 127, 130, 132, 134, or 136.

5. An antibody that selectively binds a polypeptide drawn from the group consisting of: SEQ ID NO: 2, 5, 7, 9, 11, 14, 16, 18, 20, 22, 24, 27, 29, 32, 34, 36, 38, 40, 42, 44, 46, 48, 51, 54, 56, 59, 61, 63, 65, 68, 70, 73, 75, 77, 80, 82, 85, 88, 91, 93, 95, 97, 99, 101, 103, 105, 107, 109, 111, 113, 115, 117, 19, 121, 123, 125, 127, 130, 132, 134, and 136.

6. An oligonucleotide that inhibits the expression of a nucleic acid molecule that encodes an amino acid sequence drawn from the group consisting of: SEQ ID NO: 2, 5, 7, 9, 11, 14, 16, 18, 20, 22, 24, 27, 29, 32, 34, 36, 38, 40, 42, 44, 46, 48, 51, 54, 56, 59, 61, 63, 65, 68, 70, 73, 75, 77, 80, 82, 85, 88, 91, 93, 95, 97, 99, 101, 103, 105, 107, 109, 111, 113, 115, 117, 119, 121, 123, 125, 127, 130, 132, 134, and 136.
